(19) United States
(12) Patent Application Publication
(10) Pub. No.: US 2020/0331978 A1
(43) Pub. Date: Oct. 22, 2020

(54) Compositions and Methods for Treating or Preventing Endocrine FGF23-Linked Diseases

(71) Applicants: YALE UNIVERSITY, New Haven, CT (US); ICAHN SCHOOL OF MEDICINE AT MOUNT SINAI, New York, NY (US)

(72) Inventors: Joseph Schlessinger, Woodbridge, CT (US); Sangwon Lee, Branford, CT (US); Avner Schlessinger, New York, NY (US); Man-Un Ung, New York, NY (US)

(21) Appl. No.: 16/772,100

(22) PCT Filed: Dec. 12, 2018
(86) PCT No.: PCT/US2018/065236

§ 371 Date: Jun. 11, 2020

Related U.S. Application Data
(60) Provisional application No. 62/598,273, filed on Dec. 13, 2017.

(51) Int. Cl. C07K 14/50

(52) U.S. Cl. CPC C07K 14/50; A61K 38/00

(57) ABSTRACT

The present invention provides compositions and methods that are useful in treating or preventing endocrine FGF-related diseases or disorders, such as but not limited to dysfunctional phosphate homeostasis and chronic kidney disease (CKD).

### CROSS-REFERENCE TO RELATED APPLICATION

The present application claims priority under 35 U.S.C. § 119(e) to U.S. Provisional Application No. 62/598,273, filed Dec. 13, 2017, which application is incorporated herein by reference in its entirety.

### BACKGROUND OF THE INVENTION

Cellular signaling initiated by fibroblast growth factors (FGFs) controls important physiological processes during normal embryonic development and homeostasis in adult animals. Accordingly, a variety of diseases are caused by genetic disruption or aberrant regulation of FGF-dependent cell signaling pathways. The 22 members of the FGF family stimulate their cellular responses by binding to the extracellular domains of four members of the fibroblast growth factor receptors (FGFRs), which are a family of receptor tyrosine kinases (RTKs).

Canonical FGFs activate FGFRs through paracrine or autocrine mechanisms, in a process that requires the action of an FGF ligand together with heparan sulfate proteoglycans (HSPG) that function as critical co-receptors for FGFs. This requirement for HSPGs distinguishes FGFRs from most other RTKs, which are typically activated directly by specific growth factor binding to the extracellular domains of a cognate receptor. Receptor dimerization is crucial for FGFR activation as with other RTKs. In contrast with other growth factors such as EGF and PDGF, however, canonical FGFs can stimulate FGFR dimerization only when bound to HSPGs. FGFR dimerization leads to kinase activation and trans-phosphorylation of specific tyrosine residues in the receptor cytoplasmic domain. This, in turn, triggers stimulation of multiple signaling pathways, either through direct association of signaling molecules with activated FGFR or through indirect interactions mediated by closely associated docking proteins such as FRS2 and Gab1, specialized in recruiting unique complements of signaling proteins.

Endocrine FGFs FGF19, FGF21 and FGF23 are members of the FGF family that function as circulating hormones regulating a variety of critical metabolic functions in different cells and tissues. FGF23 plays an important role in the control of phosphate homeostasis, FGF19 inhibits bile acid synthesis, and FGF21 regulates energy expenditure and other critical metabolic processes. The target organs of FGF23 are kidney and parathyroid FGF23 binding stimulates urinary phosphate excretion and decreases parathyroid hormone levels, respectively. Unlike canonical FGFs that require HSPG to activate FGFRs, endocrine FGFs do not have this requirement, but instead are specifically dependent on Klotho co-receptors for FGFR activation.

There are two Klothos, encoded by different genes. α-Klotho (KLA) is required for FGF23-dependent signaling, and β-Klotho (KLB) is essential for FGF19- or FGF21-dependent signaling in specific tissues and organs. Although different FGFRs are expressed throughout the body, expression of Klotho proteins is limited to specific tissues—α-Klotho expression is confined to the kidney and parathyroid, whereas β-Klotho expression is limited to adipose tissue, liver, pancreas and hypothalamus. Both Klotho proteins are membrane receptors composed of an N-terminal extracellular region and a single transmembrane spanning region followed by a short cytoplasmic region. Each Klotho extracellular region contains tandem domains that share sequence similarity with the glycoside hydrolase family of enzymes. Amino acid sequence alignments indicate that one of the two catalytic amino acid residues of each of Klotho's glycoside hydrolase-like domains (GH domain) were substituted at some point in its evolution, indicating that Klotho's GH domains are deficient in enzymatic activity and can be defined as pseudo-enzymes. However, several reports have suggested that α-Klotho has some detectable enzymatic activity.

There is a need in the art to identify compositions and methods that can be used to modulate (e.g. inhibit or stimulate) the activity of FGF receptors and the signaling pathways activated by endocrine FGFs. In certain embodiments, these compositions and methods are useful in treating, ameliorating and/or preventing diseases (such as, but not limited to, dysfunctional phosphate homeostasis) associated with endocrine FGFs. The present invention fulfills these needs.

### BRIEF SUMMARY OF THE INVENTION

The invention provides a non-natural soluble construct that prevents or minimizes the binding of a FGF receptor (FGFR) and/or FGF23 to α-Klotho. In certain embodiments, the construct prevents FGFR activation. The invention further provides a soluble construct comprising a FGF23 polypeptide that binds to α-Klotho more tightly than wild-type FGF23 and elicits enhanced biological activity as compared to wild-type FGF23. The invention further provides a construct that simultaneously binds to an exposed epitope on FGF23_{CT} and an exposed epitope on α-Klotho in a FGF23_{CT}-α-Klotho complex, stabilizing the FGF23_{CT}-α-Klotho complex formation and eliciting enhanced biological activity as compared to wild-type FGF23. The invention further provides a construct comprising a FGF23 polypeptide fused to a α-Klotho binder, wherein the construct has FGF23 stimulatory activities. The invention further provides a method of treating and/or preventing endocrine FGF-related diseases or disorders in a mammal in need thereof.

In certain embodiments, the α-Klotho is on the surface of a mammal's cell.

In certain embodiments, the construct is an antibody, nanobody, recombinant protein, and/or small molecule. In certain embodiments, the construct is an antibody and/or a recombinant peptide. In certain embodiments, the antibody is selected from the group consisting of a polyclonal antibody, monoclonal antibody, humanized antibody, synthetic antibody, heavy chain antibody, human antibody, biologically active fragment of an antibody, and any combinations thereof.

In certain embodiments, the construct recognizes and binds to at least one amino acid residue of FGF23 that binds to α-Klotho, thus preventing FGF23 binding to α-Klotho. In certain embodiments, the construct recognizes and/or binds to at least one amino acid residue of α-Klotho that binds to FGF23, thus preventing α-Klotho binding to FGF23. In certain embodiments, the construct recognizes and/or binds to one or more amino acids within the amino acid residues 377-925 in α-Klotho (SEQ ID NO:1).

In certain embodiments, the construct recognizes and/or binds to one or more amino acids selected from the group consisting of F377, Q378, E390, S391, P392, W417, F418, V419, S420, K429, Y432, Y433, K436, N530, Q639, P640, M641, A642, P643, N688, E689, P690, T692, Q731, D733, V752, D756, S807, Y809, I812, D815, L828, V830, Q831, E832, M833, T834, I836, V845, S872, Y915, S916, A922, P923, and F925 of SEQ ID NO:1.

In certain embodiments, the construct recognizes and binds to at least one amino acid residue of α-Klotho that binds to a FGFR, thus preventing α-Klotho binding to the FGFR. In certain embodiments, the construct recognizes and/or binds to one or more amino acids within the extracellular region of human α-Klotho (amino acid residues 34-981 of SEQ ID NO:1). In certain embodiments, the construct recognizes and/or binds to one or more amino acids within the fragment of the extracellular region of human α-Klotho comprising amino acid residues 534-571 of SEQ ID NO:1.

In certain embodiments, the construct comprises a FGF23 polypeptide that is capable of binding to and sequestering α-Klotho on the surface of a mammal's cell.

In certain embodiments, the construct comprises amino acid residues 180-251 of SEQ ID NO:3 (FGF23_{CT}), or a fragment thereof.

In certain embodiments, the construct comprises a α-Klotho polypeptide that is capable of binding to and sequestering FGF23.

In certain embodiments, the α-Klotho polypeptide comprises the extracellular region of human α-Klotho (amino acids 34-981 of SEQ ID NO:1), or a fragment thereof.

In certain embodiments, the α-Klotho polypeptide comprises amino acids 377-925 of SEQ ID NO:1, or a fragment thereof.

In certain embodiments, the construct comprises a α-Klotho polypeptide that is capable of binding to a FGFR.

In certain embodiments, the construct comprises the extracellular region of human α-Klotho (amino acid residues 34-981 of SEQ ID NO:1), or a fragment thereof.

In certain embodiments, the construct comprises amino acid residues 534-571 of SEQ ID NO:1, or a fragment thereof.

In certain embodiments, the construct is fused to a stability enhancing domain. In certain embodiments, the stability enhancing domain comprises albumin, thioredoxin, glutathione S-transferase, and/or a Fc region of an antibody. In certain embodiments, the polypeptide and the stability enhancing domain are linked through a polypeptide comprising about 1-18 amino acids.

The invention further provides a soluble construct comprising a FGF23 polypeptide that binds to α-Klotho more tightly than wild-type FGF23 and elicits enhanced biological activity as compared to wild-type FGF23.

In certain embodiments, the FGF23 polypeptide has at least one mutation in its C-terminal domain.

In certain embodiments, the method comprises administering to the mammal a therapeutically effective amount of a construct that modulates interaction of FGF23 with α-Klotho on the surface of a cell of the mammal.

In certain embodiments, the construct prevents or minimizes binding of FGF23 to α-Klotho on the surface of the mammal's cell.

In certain embodiments, the disease or disorder includes hypophosphatemia and/or tumor-induced osteomalacia.

In certain embodiments, the construct binds more tightly than wild-type FGF23 to α-Klotho on the surface of the mammal's cell.

In certain embodiments, the mammal is human.

In certain embodiments, the construct is administered by an administration route selected from the group consisting of inhalational, oral, rectal, vaginal, parenteral, intracranial, topical, transdermal, pulmonary, intranasal, buccal, ophthalmic, intrathecal, and intravenous.

In certain embodiments, the construct is formulated for administration by an administration route selected from the group consisting of inhalational, oral, rectal, vaginal, parenteral, intracranial, topical, transdermal, pulmonary, intranasal, buccal, ophthalmic, intrathecal, and intravenous.

In certain embodiments, the mammal is further administered at least one additional drug that treats or prevents the disease and/or disorder.

In certain embodiments, the construct and the at least one additional drug are co-administered. In certain embodiments, the construct and the at least one additional drug are co-formulated.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A-1C illustrate amino acid sequence alignment (FIG. 1A) of the C-terminal regions of endocrine FGFs and (FIG. 1B) and amino acid sequence alignment of the extracellular region of β-Klotho (sKLB) with the extracellular region of α-Klotho (sKLA).

FIG. 1A: Asp-Pro motif conserved among all endocrine FGFs is highlighted in light blue (dark grey), and Ser-Pro-Ser motif in FGF19 and FGF21 is highlighted in yellow (light grey). FGF21(183-209), SEQ ID NO:6; FGF19(189-216), SEQ ID NO:7, FGF23(180-205), SEQ ID NO:8. FIGS. 1B-1C: Alignment shown for sKLB (residues 34-980 of SEQ ID NO:2) and sKLA (residues 53-995 of SEQ ID NO:1). Amino acid residues in sKLB interacting with FGF21_{CT} that are identified from the crystal structure and the corresponding amino acid residues in sKLA are highlighted in boxes on the corresponding sequences.

FIGS. 2A-2C illustrate a non-limiting homology model of sKLA. FIG. 2A: Comparison between the crystal structure of sKLB (top panel, ribbon representation) in complex with FGF21_{CT} (stick representation) and the homology model of sKLA (bottom panel, ribbon representation). In FIGS. 2B-2C, the structures are overlaid to each other, and the key residues in (FIG. 2B) site-1 or (FIG. 2C) site-2 that binds to FGF21_{CT} are indicated. Site-1 of sKLA contains hydrophobic surfaces that may support residues from either FGF21_{CT}, FGF19_{CT} or FGF23_{CT} in this area, while site-2 of sKLA contains residues with different properties that only accommodate FGF23_{CT}.

FIGS. 3A-3B illustrate non-limiting models of a crystal structure of sKLB in complex with FGF21_{CT} (stick representation) (FIG. 3A) and a homology model of sKLA (FIG. 3B). Electrostatic potential, ranged between −6 K_{b}T/e_{c} (red) and +6 K_{b}T/e_{c} (blue), are indicated on the surface representation of each model. Negatively-charged surface area on sKLA that may prevent binding of FGF21 and FGF19 is highlighted (FIG. 3B, dotted line).

FIGS. 4A-4B illustrate a non-limiting representation of sKLA models, highlighting areas to which inhibitors can bind (dotted lines). See Table 1 for complete list of amino acid residues.

### DETAILED DESCRIPTION OF THE INVENTION

The present invention relates in one aspect to the discovery that α-Klotho is the primary cell-surface receptor for FGF23 In one aspect, the invention provides compositions and methods that are useful in treating or preventing endocrine FGF-related diseases or disorders, such as for example diseases or disorders associated with dysregulated phosphate homeostasis.

FGF23 is a bone-derived hormone that play as an important physiological regulator of renal Pi excretion. Transgenic mice that overexpresses FGF23 develop hypophosphatemia, whereas FGF23-knockout mice develop hyperphosphatemia, which can be reversed by systemic injection of human FGF23.

Importantly, these in vivo actions of FGF23 require the presence of α-Klotho. Injection of FGF23 into α-Klotho-knockout mice or FGF23/α-Klotho-double knockout mice did not affect the serum phosphate level. Like other endocrine FGFs, FGF23 exhibits isoform specificity for FGFRs—it binds and activates IIIc isoform of FGFR1 and FGFR3, as well as FGFR4 which only exhibits a single isoform.

FGF23 is associated with a number of human diseases related to dysregulation of phosphate metabolism. X-linked hypophosphatemia (XLH) is an inherited disorder where PHEX (phosphate regulating gene with homologies to endopeptidases located on the X chromosome) contains loss-of-function mutation, and the consequence of this mutation is the elevation of circulating FGF23. Similarly, increased level of FGF23 was observed in autosomal recessive hypophosphatemic rickets 1 (ARHR1) or autosomal recessive hypophosphatemic rickets 2 (ARHR2) patients that carries mutations in DMP-1 or ENPP-1, respectively. In autosomal dominant hypophosphatemic rickets (ADHR), gain-of-function mutations in FGF23, R176Q and/or R179Q, prevent natural proteolytic cleavages at these sites to make two inactive fragments of FGF23. Without wishing to be limited by any theory, such cleavage can represent a mechanism of down-regulation. Cancers harboring tumors that produce high levels of FGF23 lead to tumor-induced osteomalacia (TIO), which can be reversed by surgical removal of the tumors secreting high FGF23 levels. While increased activities of FGF23 are observed in patients with disorders mentioned above, reduced activity of FGF23 has been also found in patients of hyperphosphosphatemic familial tumoral calcinosis (HFTC). A homozygous loss-of-function mutation in KLA, H193R, was also found in a HFTC patient.

The two Klotho proteins (α-Klotho and β-Klotho) are type I membrane proteins composed of a large extracellular region, a transmembrane helix and a small intracellular region. FGF23 binds specifically to α-Klotho, and FGF19 and FGF21 bind specifically to 3-Klotho. Complex formation with Klotho proteins is mediated by the C-terminal tails of endocrine FGFs. The extracellular regions of Klotho proteins contain two tandem domains with sequence homology to enzymes with glycoside hydrolases activity. The crystal structure of the extracellular region of β-Klotho (sKLB) shows that the two tandem glycoside hydrolase-like domains are connected by an unstructured and flexible linker. Each of the glycoside hydrolase-like domains in sKLB retain only one of the two glutamic acid residues required for the hydrolysis of oligosaccharide substrates, rendering sKLB as inactive enzyme (pseudo-glycoside hydrolase). The crystal structure of β-Klotho in complex with the C-terminal tail (FGF21_{CT}) shows that ligand binding is mediated by two distinct binding sites located in each of the glycoside hydrolase-like domains that are separated by approximately 30 Å. The occupied ligand adopts several turns that are connected by multiple intramolecular hydrogen bonds within the FGF21_{CT} molecule resulting in primarily hydrophobic interactions with site-1, located in domain 1 of β-Klotho. By contrast, site-2 located on domain 2 of 3-Klotho binds to a “sugar-mimicking” Ser-Pro-Ser motif on FGF21_{CT} through specific interactions between the glutamic acid that plays an important catalytic role in cleaving sugars in active glycoside hydrolases. Without wishing to be limited by any theory, similarity between FGF21_{CT}-binding region in sKLB and oligosaccharide-binding region in glycoside hydrolases suggests that the extracellular region of Klotho family of proteins evolved from an enzyme that cleaves sugars.

In one aspect, the present invention describes binding sites, epitopes and amino acid sequences of α-Klotho that can be occupied with antibodies, small molecules, and other type of antagonists for treatment of disorders caused by dysregulation of phosphate metabolism.

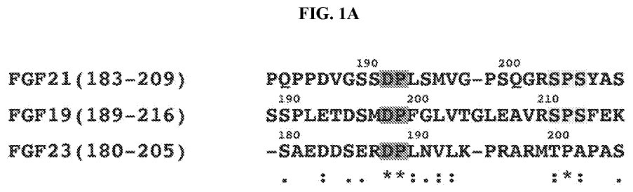

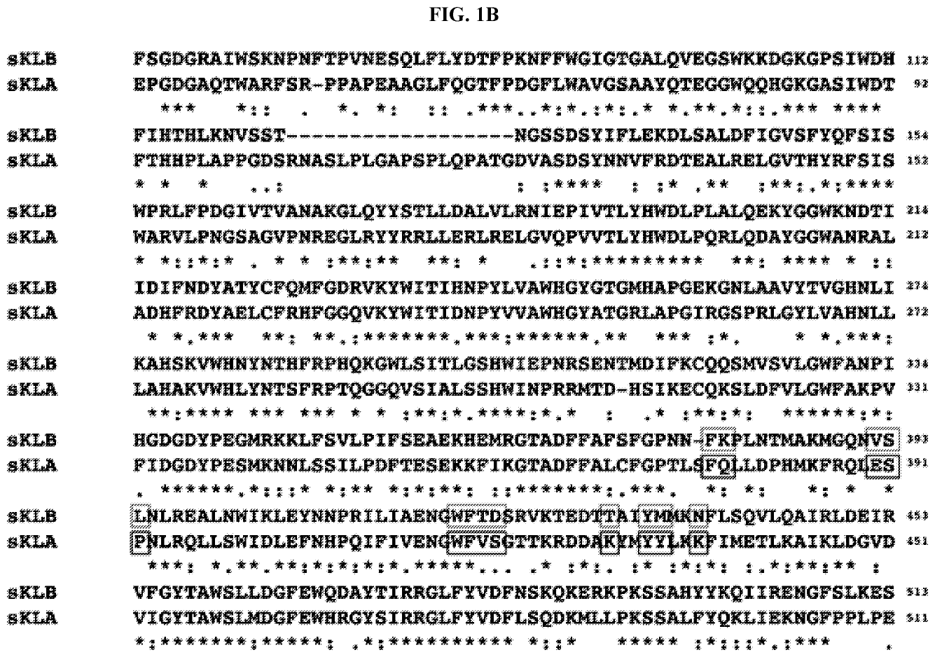

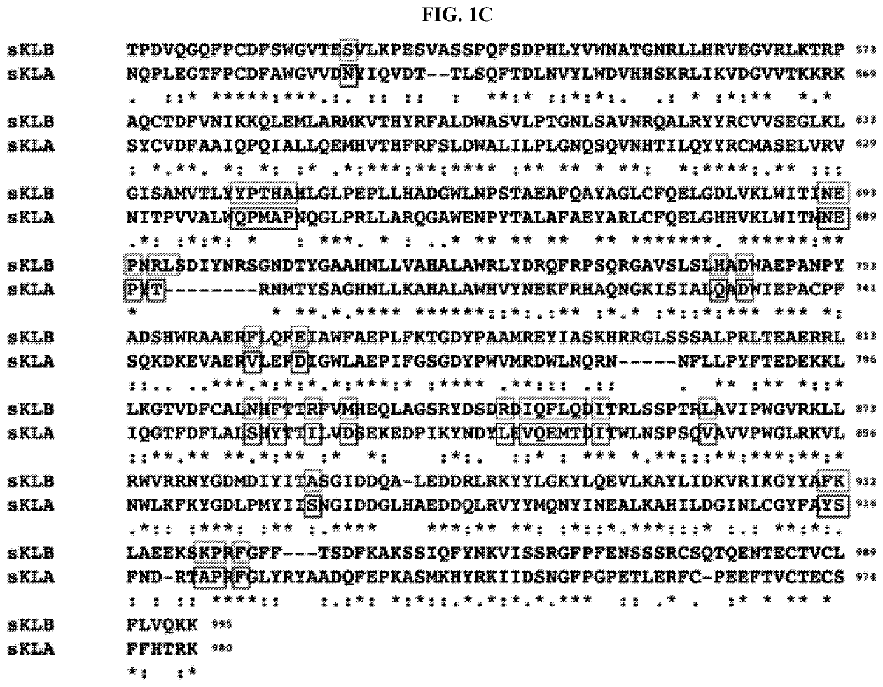

As demonstrated herein, amino acid sequence alignments suggest a conserved binding mode for endocrine FGFs. FIG. 1A shows an alignment of amino acid sequences of the C-terminal regions of FGF21, FGF19, and FGF23. The sequence alignment reveals close sequence similarity between the C-terminal tails of FGF21 and FGF19, which is consistent with the similar binding characteristics of FGF21 and FGF19 and their isolated C-terminal regions to β-Klotho. The sugar-mimicking motif in FGF21 (S205-P206-S207) is conserved in FGF19 (S211-P212-S213). Also highlighted is the sequence D192-P193 in the region of FGF21_{CT} that stabilizes intramolecular hydrogen bonds that maintain a turn in the bound configuration of FGF21_{CT}. This sequence is conserved in FGF19 (D198-P199), suggesting in non-limiting embodiments that similar intramolecular interactions responsible for mediating consecutive turns in FGF19_{CT} can also similarly bind to β-Klotho. Moreover, since many of the intramolecular interactions within FGF21_{CT} bound to β-Klotho take place between main chain atoms (as observed in typical β-turn structures), only few key amino acids in the sequence such as D198-P199 can be sufficient for generating a similar multi-turn elements in FGF19_{CT} as observed in the crystal structure of FGF21_{CT} bound to β-Klotho.

By contrast, the C-terminal region of FGF23 is longer (72 amino acids) than the C-terminal regions of FGF21 (38 amino acids) or FGF19 (39 amino acids). Amino acids S180-S205 of the C-terminal region of FGF23 are critical for mediating interaction between FGF23 with α-Klotho. The C-terminal region of FGF23 exhibits weak sequence similarities to the sequences of C-terminal regions of FGF21 and FGF19, which is consistent with the binding specificity of FGF23 to α-Klotho and not to β-Klotho. However, as highlighted in FIG. 1A, amino acids critical for maintaining the multi-turn element Asp-Pro are conserved in FGF23. Without wishing to be limited by any theory, this indicates that FGF23 can also contain a multi-turn element in the region of FGF23_{CT} that binds to α-Klotho. The sugar-mimicking sequence Ser-Pro-Ser in FGF21 or FGF19 is absent from FGF23. Without wishing to be limited by any theory, this indicates that alternative interactions can take between amino acid residues of FGF23_{CT} that bind to domain 2 of α-Klotho from those seen in the crystal structure FGF21_{CT} bound to sKLB.

Alignment of amino acid sequences of extracellular regions of β-Klotho (sKLB) and α-Klotho (sKLA) (as shown in FIGS. 1B-1C), on the other hand, reveal high sequence similarity (48.9% identity). Highlighted in green are the amino acids identified in crystal structure of sKLB that interact with FGF21_{CT}, and highlighted in red are corresponding amino acids of sKLA. Most, but not all, of these amino acids were substituted by other amino acids in α-Klotho, raising the possibility the substituted amino acids may play a role in specific recognition of FGF23 by α-Klotho.

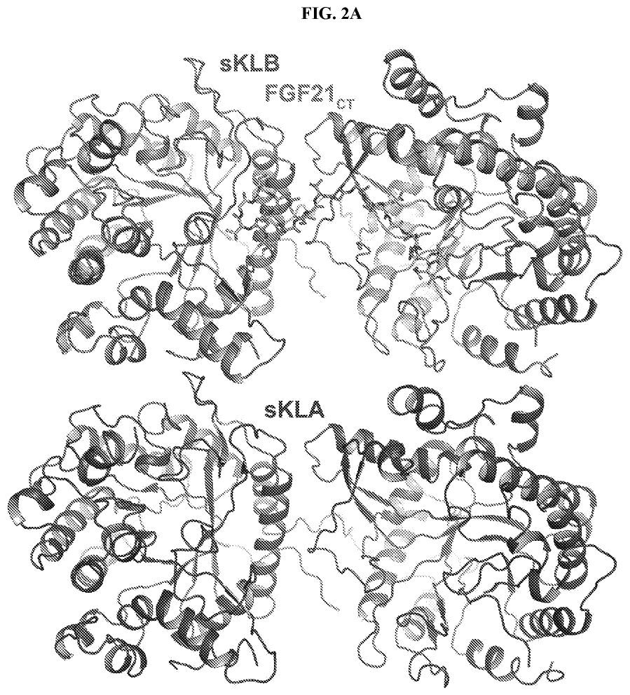

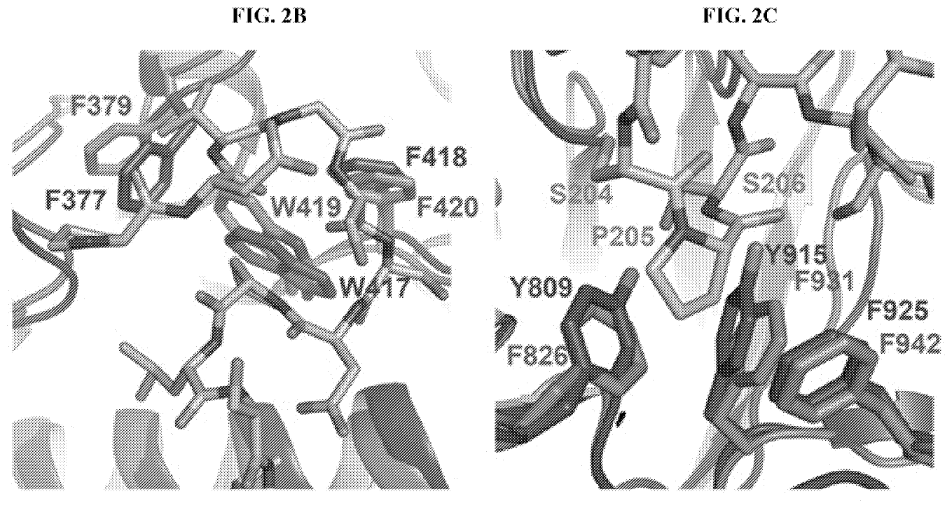

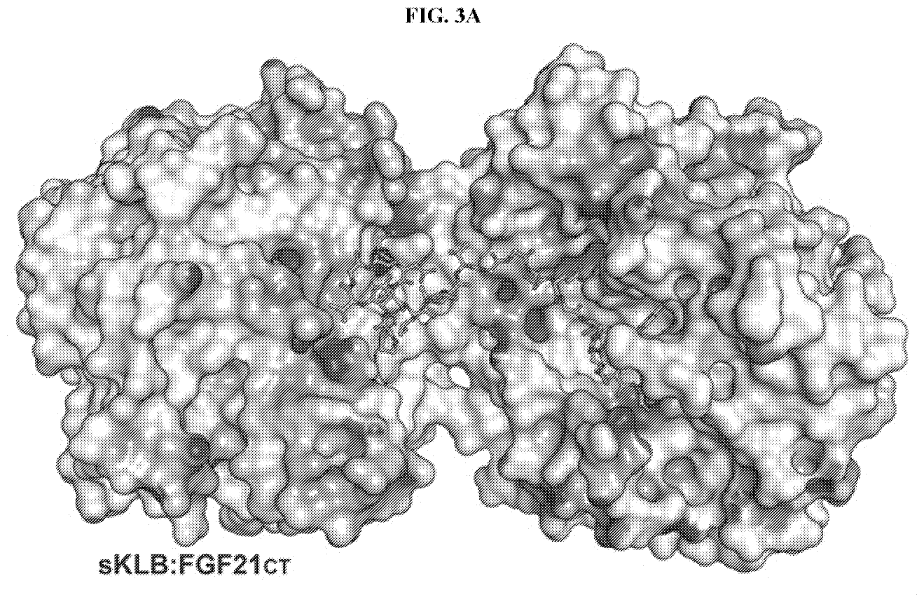

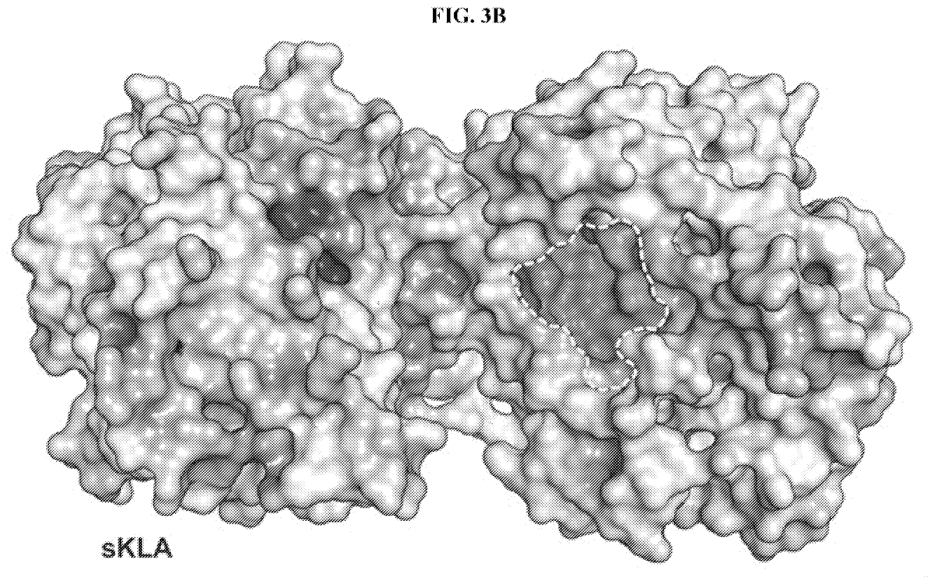

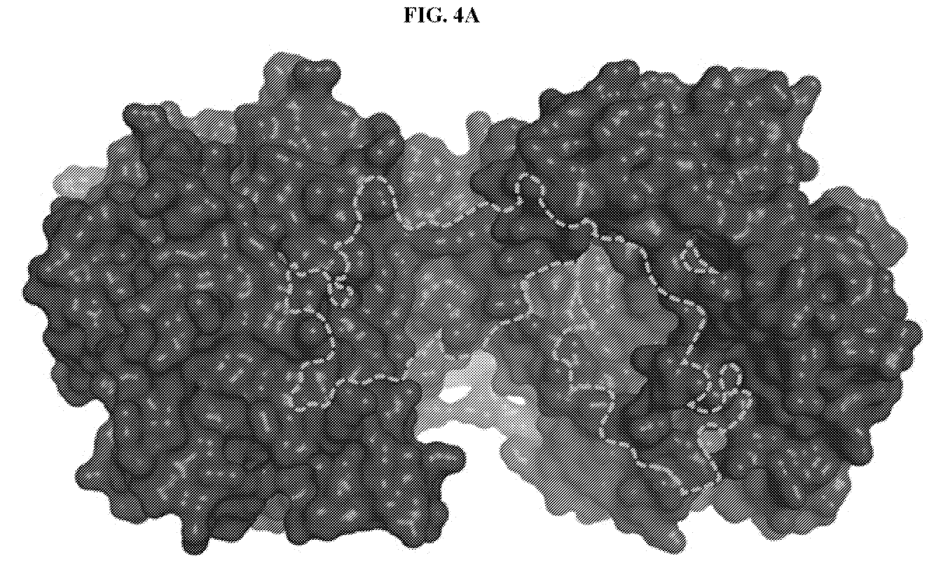

As demonstrated herein, a homology model of α-Klotho reveals binding sites for FGF23 and unique interactions between FGF23 and α-Klotho. High sequence similarity in the amino acid sequences of sKLB and sKLA suggests that a homology model of sKLA structure can be built based on the crystal structure of sKLB with reasonable accuracy (FIGS. 2A-2C). The overall folds, as seen in FIG. 2A, of two proteins are identical. However, comparison of amino acid residues in sKLB that interact with FGF21_{CT} with corresponding amino acids in sKLA reveals two important features: (1) amino acids in domain 1 that are critical for hydrophobic interactions with the ligand are conserved in β-Klotho and α-Klotho as shown in FIG. 2B; (2) phenylalanine residues in domain 2 of sKLB that are critical for maintaining hydrophobic interaction with the sugar-mimicking motif, Ser-Pro-Ser, are substituted by tyrosine residues in α-Klotho as depicted in FIG. 2C. These two features of the sKLA model are consistent with the sequence alignment of amino acids of C-terminal region of ligands: the putative multi-turn element in FGF23_{CT} can be supported by a hydrophobic surface created by F377, W417, and F418 in sKLA model. Substitution of phenylalanine residues in sKLB (that supports S-P-S in FGF21_{CT}) by tyrosine residues in sKLA can create increased negative charged in site-2, that will prevent hydroxyl groups in S-P-S motif from approaching the polar pocket in sKLA as highlighted in FIGS. 3A-3B. In certain embodiments, amino acids residues that form site-1 can function as a “promiscuous” hydrophobic surface for ligand binding, while amino acids located in site-2 can represent a pocket playing a critical role in determining endocrine FGF binding selectivity.

### Definitions

As used herein, each of the following terms have the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, crystallography, chemistry, and computational modeling are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of 20% or 10%, ±5%, 1%, or 0.1% from the specified value, as such variations are appropriate to perform the disclosed methods.

As used herein, the term “α-Klotho” or “KLA” refers to the protein of amino sequence of SEQ ID NO:1:

```
                10 20 30 40 50
MPASAPPRRP RPPPPSLSLL LVLLGLGGRR LRAEPGDGAQ TWARFSRPPA
                60 70 80 90 100
PEAAGLFQGT FPDGFLWAVG SAAYQTEGGW QQHGKGASIW DTFTHHPLAP
              110 120 130 140 150
PGDSRNASLP LGAPSPLQPA TGDVASDSYN NVFRDTEALR ELGVTHYRFS
              160 170 180 190 200
ISWARVLPNG SAGVPNREGL RYYRRLLERL RELGVQPVVT LYHWDLPQRL
              210 220 230 240 250
QDAYGGWANR ALADHFRDYA ELCFRHFGGQ VKYWITIDNP YVVAWHGYAT
              260 270 280 290 300
GRLAPGIRGS PRLGYLVAHN LLLAHAKVWH LYNTSFRPTQ GGQVSIALSS
              310 320 330 340 350
HWINPRRMTD HSIKECQKSL DFVLGWFAKP VFIDGDYPES MKNNLSSILP
              360 370 380 390 400
DFTESEKKFI KGTADFFALC FGPTLSFQLL DPHMKFRQLE SPNLRQLLSW
              410 420 430 440 450
IDLEFNHPQI FIVENGWFVS GTTKRDDAKY MYYLKKFIME TLKAIKLDGV
              460 470 480 490 500
DVIGYTAWSL MDGFEWHRGY SIRRGLFYVD FLSQDKMLLP KSSALFYQKL
              510 520 530 540 550
IEKNGFPPLP ENQPLEGTFP CDFAWGVVDN YIQVDTTLSQ FTDLNVYLWD
              560 570 580 590 600
VHHSKRLIKV DGVVTKKRKS YCVDFAAIQP QIALLQEMHV THFRFSLDWA
              610 620 630 640 650
LILPLGNQSQ VNHTILQYYR CMASELVRVN ITPVVALWQP MAPNQGLPRL
              660 670 680 690 700
LARQGAWENP YTALAFAEYA RLCFQELGHH VKLWITMNEP YTRNMTYSAG
              710 720 730 740 750
HNLLKAHALA WHVYNEKFRH AQNGKISIAL QADWIEPACP FSQKDKEVAE
              760 770 780 790 800
RVLEFDIGWL AEPIFGSGDY PWVMRDWLNQ RNNFLLPYFT EDEKKLIQGT
              810 820 830 840 850
FDFLALSHYT TILVDSEKED PIKYNDYLEV QEMTDITWLN SPSQVAVVPW
              860 870 880 890 900
GLRKVLNWLK FKYGDLPMYI ISNGIDDGLH AEDDQLRVYY MQNYINEALK
              910 920 930 940 950
AHILDGINLC GYFAYSFNDR TAPRFGLYRY AADQFEPKAS MKHYRKIIDS
              960 970 980 990 1000
NGFPGPETLE RFCPEEFTVC TECSFFHTRK SLLAFIAFLF FASIISLSLI
            1010
FYYSKKGRRS YK
```

As used herein, the extracellular domain of α-Klotho (sKLA) corresponds to the amino acid residues 34-981 of SEQ ID NO:1.

As used herein, the term “β-Klotho” or “KLB” refers to the protein of amino sequence of SEQ ID NO:2:

```
                10 20 30 40 50
MKPGCAAGSP GNEWIFFSTD EITTRYRNTM SNGGLQRSVI LSALILLRAV
                60 70 80 90 100
TGFSGDGRAI WSKNPNFTPV NESQLFLYDT FPKNFFWGIG TGALQVEGSW
              110 120 130 140 150
KKDGKGPSIW DHFIHTHLKN VSSTNGSSDS YIFLEKDLSA LDFIGVSFYQ
              160 170 180 190 200
FSISWPRLFP DGIVTVANAK GLQYYSTLLD ALVLRNIEPI VTLYHWDLPL
              210 220 230 240 250
ALQEKYGGWK NDTIIDIFND YATYCFQMFG DRVKYWITIH NPYLVAWHGY
              260 270 280 290 300
GTGMHAPGEK GNLAAVYTVG HNLIKAHSKV WHNYNTHFRP HQKGWLSITL
              310 320 330 340 350
GSHWIEPNRS ENTMDIFKCQ QSMVSVLGWF ANPIHGDGDY PEGMRKKLFS
              360 370 380 390 400
VLPIFSEAEK HEMRGTADFF AFSFGPNNFK PLNTMAKMGQ NVSLNLREAL
              410 420 430 440 450
NWIKLEYNNP RILIAENGWF TDSRVKTEDT TAIYMMKNFL SQVLQAIRLD
              460 470 480 490 500
EIRVFGYTAW SLLDGFEWQD AYTIRRGLFY VDFNSKQKER KPKSSAHYYK
              510 520 530 540 550
QIIRENGFSL KESTPDVQGQ FPCDFSWGVT ESVLKPESVA SSPQFSDPHL
              560 570 580 590 600
YVWNATGNRL LHRVEGVRLK TRPAQCTDFV NIKKQLEMLA RMKVTHYRFA
              610 620 630 640 650
LDWASVLPTG NLSAVNRQAL RYYRCVVSEG LKLGISAMVT LYYPTHAHLG
              660 670 680 690 700
LPEPLLHADG WLNPSTAEAF QAYAGLCFQE LGDLVKLWIT INEPNRLSDI
              710 720 730 740 750
YNRSGNDTYG AAHNLLVAHA LAWRLYDRQF RPSQRGAVSL SLHADWAEPA
              760 770 780 790 800
NPYADSHWRA AERFLQFEIA WFAEPLFKTG DYPAAMREYI ASKHRRGLSS
              810 820 830 840 850
SALPRLTEAE RRLLKGTVDF CALNHFTTRF VMHEQLAGSR YDSDRDIQFL
              860 870 880 890 900
QDITRLSSPT RLAVIPWGVR KLLRWVRRNY GDMDIYITAS GIDDQALEDD
              910 920 930 940 950
RLRKYYLGKY LQEVLKAYLI DKVRIKGYYA FKLAEEKSKP RFGFFTSDFK
              960 970 980 990 1000
AKSSIQFYNK VISSRGFPFE NSSSRCSQTQ ENTECTVCLF LVQKKPLIFL
            1010 1020 1030 1040
GCCFFSTLVL LLSIAIFQRQ KRRKFWKAKN LQHIPLKKGK RVVS
```

As used herein, the extracellular domain of β-Klotho (sKLB) corresponds to the amino acid residues 53-983 of SEQ ID NO:2.

The term “antibody,” as used herein, refers to an immunoglobulin molecule that specifically binds with an antigen. Antibodies can be intact immunoglobulins derived from natural sources or from recombinant sources, and can be immunoreactive portions of intact immunoglobulins. Antibodies are typically tetramers of immunoglobulin molecules. The 50 antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, scFv antibodies, single-domain antibodies such as sdAb (either VL or VH), such as camelid antibodies (Riechmann, 1999, J. Immunol. Meth. 231:25-38), camelid VHH domains, composed of either a VL or a VH domain that exhibit sufficient affinity for the target, and multispecific antibodies formed from antibody fragments such as a bivalent fragment comprising two Fab fragments linked by a disulfide bridge at the hinge region, and an isolated complementarity-determining region (CDR) or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger & Hudson, 2005, Nature Biotech. 23:1126-1136). Antigen binding fragments can also be grafted into scaffolds based on polypeptides such as a fibronectin type III (Fn3) (U.S. Pat. No. 6,703,199, which describes fibronectin polypeptide minibodies). The antibody fragment also includes a human antibody or a humanized antibody or a portion of a human antibody or a humanized antibody.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“Antisense” refers particularly to the nucleic acid sequence of the non-coding strand of a double stranded DNA molecule encoding a polypeptide, or to a sequence which is substantially homologous to the non-coding strand. As defined herein, an antisense sequence is complementary to the sequence of a double stranded DNA molecule encoding a polypeptide. It is not necessary that the antisense sequence be complementary solely to the coding portion of the coding strand of the DNA molecule. The antisense sequence may be complementary to regulatory sequences specified on the coding strand of a DNA molecule encoding a polypeptide, which regulatory sequences control expression of the coding sequences.

By the term “applicator,” as the term is used herein, is meant any device including, but not limited to, a hypodermic syringe, a pipette, and the like, for administering the compounds and compositions of the invention.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids, that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene. A “coding region” of an mRNA molecule also consists of the nucleotide residues of the mRNA molecule that are matched with an anti-codon region of a transfer RNA molecule during translation of the mRNA molecule or that encode a stop codon. The coding region may thus include nucleotide residues corresponding to amino acid residues that are not present in the mature protein encoded by the mRNA molecule (e.g., amino acid residues in a protein export signal sequence).

A “constitutive” promoter is a nucleotide sequence that, when operably linked with a polynucleotide that encodes or specifies a gene product, causes the gene product to be produced in a cell under most or all physiological conditions of the cell.

As used herein, a “disease” is a state of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount” or “therapeutically effective amount” or “pharmaceutically effective amount” of a compound are used interchangeably to refer to the amount of the compound which is sufficient to provide a beneficial effect to the subject to which the compound is administered.

As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, may be referred to as encoding the protein or other product of that gene or cDNA.

As used herein “endogenous” refers to any material from or produced inside an organism, cell, tissue or system. As used herein, the term “exogenous” refers to any material introduced from or produced outside an organism, cell, tissue or system.

The term “epitope” as used herein is defined as a small chemical molecule on an antigen that may elicit an immune response, inducing B and/or T cell responses. An antigen may have one or more epitopes. Most antigens have many epitopes; i.e., they are multivalent. In general, an epitope is roughly five amino acids and/or sugars in size. One skilled in the art understands that generally the overall three-dimensional structure, rather than the specific linear sequence of the molecule, is the main criterion of antigenic specificity and therefore distinguishes one epitope from another.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression may be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “FGF19” refers to a polypeptide of SEQ ID NO:3:

```
                10 20 30 40 50
MRSGCVVVHV WILAGLWLAV AGRPLAFSDA GPHVHYGWGD PIRLRHLYTS
                60 70 80 90 100
GPHGLSSCFL RIRADGVVDC ARGQSAHSLL EIKAVALRTV AIKGVHSVRY
              110 120 130 140 150
LCMGADGKMQ GLLQYSEEDC AFEEEIRPDG YNVYRSEKHR LPVSLSSAKQ
              160 170 180 190 200
RQLYKNRGFL PLSHFLPMLP MVPEEPEDLR GHLESDMFSS PLETDSMDPF
              210
GLVTGLEAVR SPSFEK
```

As used herein, “FGF19_{CT}” refers to a polypeptide corresponding to the amino acid residues 170-216 of SEQ ID NO:3.

As used herein, the term “FGF21” refers to a polypeptide of SEQ ID NO:4:

```
                10 20 30 40 50
MDSDETGFEH SGLWVSVLAG LLLGACQAHP IPDSSPLLQF GGQVRQRYLY
                60 70 80 90 100
TDDAQQTEAH LEIREDGTVG GAADQSPESL LQLKALKPGV IQILGVKTSR
              110 120 130 140 150
FLCQRPDGAL YGSLHFDPEA CSFRELLLED GYNVYQSEAH GLPLHLPGNK
              160 170 180 190 200
SPHRDPAPRG PARFLPLPGL PPALPEPPGI LAPQPPDVGS SDPLSMVGPS
              210
QGRSPSYAS
```

As used herein, “FGF21_{CT}” refers to a polypeptide corresponding to the amino acid residues 169-209 of SEQ ID NO:4, which in certain embodiments contains two mutations, P199G and A208E (see US20120087920, which is incorporated herein in its entirety by reference).

As used herein, the term “FGF23” refers to a polypeptide of SEQ ID NO:5:

```
                10 20 30 40 50
MLGARLRLWV CALCSVCSMS VLRAYPNASP LLGSSWGGLI HLYTATARNS
                60 70 80 90 100
YHLQIHKNGH VDGAPHQTIY SALMIRSEDA GFVVITGVMS RRYLCMDFRG
              110 120 130 140 150
NIFGSHYFDP ENCRFQHQTL ENGYDVYHSP QYHFLVSLGR AKRAFLPGMN
              160 170 180 190 200
PPPYSQFLSR RNEIPLIHFN TPIPRRHTRS AEDDSERDPL NVLKPRARMT
              210 220 230 240 250
PAPASCSQEL PSAEDNSPMA SDPLGVVRGG RVNTHAGGTG PEGCRPFAKF
I
```

As used herein, “FGF23_{CT}” refers to a polypeptide corresponding to the amino acid residues 180-251 of SEQ ID NO:5.

As used herein, the term “heavy chain antibody” or “heavy chain antibodies” comprises immunoglobulin molecules derived from camelid species, either by immunization with an antigen and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer of ten subunits in length) of the positions in two sequences are homologous, the two sequences are 50% homologous; if 90% of the positions (e.g., 9 of 10), are matched or homologous, the two sequences are 90% homologous. By way of example, the DNA sequences 5′-ATTGCC-3′ and 5′-TATGGC-3′ share 50% homology.

As used herein, the term “homology modeling” refers to predicting the three-dimensional structure of a target protein based on the alignment of its sequence to one or more proteins of known structure (templates). Homology modeling includes the following steps: fold assignment, target-template alignment, model building, model refinement, and model evaluation. For example, MODELLER generates homology models based on satisfaction of spatial restraints derived from various sources, including the template-target alignment and stereochemistry (Sali and Blundell, 1993, J Mol. Biol. 234:779-815).

As used herein, the term “immunoglobulin” or “Ig” is defined as a class of proteins that function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitor-urinary tracts. IgG is the most common circulating antibody. IgM is the main immunoglobulin produced in the primary immune response in most mammals. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

An “inducible” promoter is a nucleotide sequence that, when operably linked with a polynucleotide that encodes or specifies a gene product, causes the gene product to be produced in a cell substantially only when an inducer that corresponds to the promoter is present in the cell.

The terms “inhibit” and “antagonize”, as used herein, mean to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Isolated” means altered or removed from the natural state. For example, a nucleic acid or a peptide naturally present in a living animal is not “isolated,” but the same nucleic acid or peptide partially or completely separated from the co-existing materials of its natural state is “isolated.” An isolated nucleic acid or protein may exist in substantially purified form, or may exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences that are normally adjacent to the fragment, i.e., the sequences adjacent to the fragment in a genome in which it naturally occurs. The term also applies to nucleic acids that have been substantially purified from other components which naturally accompany the nucleic acid, i.e., RNA or DNA or proteins, which naturally accompany it in the cell. The term therefore includes, for example, a recombinant DNA that is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA that is part of a hybrid gene encoding additional polypeptide sequence.

As used herein, the term “modulate” is meant to refer to any change in biological state, i.e. increasing, decreasing, and the like. For example, the term “modulate” may be construed to refer to the ability to regulate positively or negatively the expression, stability or activity of a target protein, including but not limited to transcription of a target protein mRNA, stability of a target protein mRNA, translation of a target protein mRNA, target protein stability, target protein post-translational modifications, target protein activity, or any combination thereof. Further, the term modulate may be used to refer to an increase, decrease, masking, altering, overriding or restoring of activity, including but not limited to, target protein activity.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of a composition includes, e.g., subcutaneous (s.c.), intravenous (i.v.), intramuscular (i.m.), or intrasternal injection, or infusion techniques.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary, intracranial and topical administration.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

“Pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer. The term “protein” typically refers to large polypeptides. The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus. As used herein, a “peptidomimetic” is a compound containing non-peptidic structural elements that is capable of mimicking the biological action of a parent peptide. A peptidomimetic may or may not comprise peptide bonds.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease. Disease and disorder are used interchangeably herein.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers may be labeled with, e.g., chromogenic, radioactive, or fluorescent moieties and used as detectable moieties.

“Probe” refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes may be labeled with, e.g., chromogenic, radioactive, or fluorescent moieties and used as detectable moieties.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements that are required for expression of the gene product. The promoter/regulatory sequence may for example be one that expresses the gene product in a tissue specific manner.

The term “recombinant DNA” as used herein is defined as DNA produced by joining pieces of DNA from different sources. The term “recombinant polypeptide” as used herein is defined as a polypeptide produced by using recombinant DNA methods.

The term “RNA” as used herein is defined as ribonucleic acid.

By the term “specifically bind” or “specifically binds,” as used herein, is meant that a first molecule (e.g., an antibody) preferentially binds to a second molecule (e.g., a particular antigenic epitope), but does not necessarily bind only to that second molecule.

As used herein, a “subject” refers to a human or non-human mammal. Non-human mammals include, for example, livestock and pets, such as ovine, bovine, porcine, canine, feline and murine mammals. In certain embodiments, the subject is human.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “tissue-specific” promoter is a nucleotide sequence that, when operably linked with a polynucleotide encodes or specified by a gene, causes the gene product to be produced in a cell substantially only if the cell is a cell of the tissue type corresponding to the promoter.

The term “transfected” or “transformed” or “transduced” as used herein refers to a process by which exogenous nucleic acid is transferred or introduced into the host cell. A “transfected” or “transformed” or “transduced” cell is one that has been transfected, transformed or transduced with exogenous nucleic acid. The cell includes the primary subject cell and its progeny.

As used herein, the term “treatment” or “treating” is defined as the application or administration of a therapeutic agent, i.e., a composition useful within the invention (alone or in combination with another pharmaceutical agent), to a subject, or application or administration of a therapeutic agent to an isolated tissue or cell line from a subject (e.g., for diagnosis or ex vivo applications), who has a disease or disorder, a symptom of a disease or disorder or the potential to develop a disease or disorder, with the purpose to cure, heal, alleviate, relieve, alter, remedy, ameliorate, improve or affect the disease or disorder, the symptoms of the disease or disorder or the potential to develop the disease or disorder. Such treatments may be specifically tailored or modified, based on knowledge obtained from the field of pharmacogenomics. An appropriate therapeutic amount in any individual case may be determined by one of ordinary skill in the art using routine experimentation.

The phrase “under transcriptional control” or “operatively linked” as used herein means that the promoter is in the correct location and orientation in relation to a polynucleotide to control the initiation of transcription by RNA polymerase and expression of the polynucleotide.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide may differ in amino acid sequence by one or more substitutions, additions, or deletions in any combination. A variant of a nucleic acid or peptide may be a naturally occurring such as an allelic variant, or may be a variant that is not known to occur naturally. Non-naturally occurring variants of nucleic acids and peptides may be made by mutagenesis techniques or by direct synthesis.

A “vector” is a composition of matter that comprises an isolated nucleic acid and that may be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Abbreviation used herein include: FGF, fibroblast growth factor; FGFR, fibroblast growth factor receptor; KLA, α-Klotho; KLB, β-Klotho; GH domain, glycoside hydrolase-like domain; HSPG, heparan sulfate proteoglycans; RMSD, root-mean-square deviation; RTK, receptor tyrosine kinase; sKLA, extracellular domain of α-Klotho; sKLB, extracellular domain of β-Klotho.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

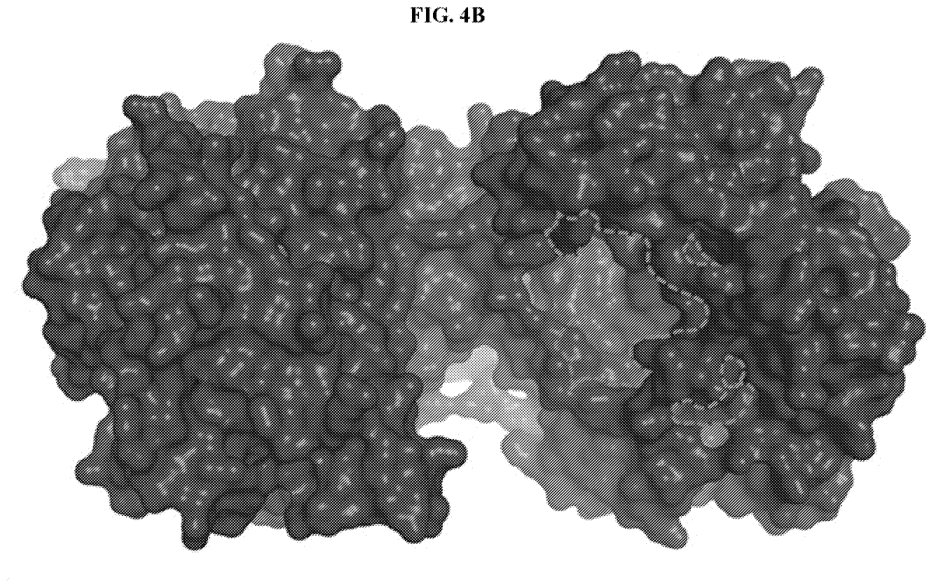

TABLE 1
Amino acid residues of α-Klotho that can be used for
inhibition studies (see also FIGS. 1B-1C, and FIGS. 4A-4B).

| | | | | | | | | |
|---|---|---|---|---|---|---|---|---|
| F377 | Q378 | E390 | S391 | P392 | W417 | F418 | V419 | S420 |
| K429 | Y432 | Y433 | K436 | N530 | Q639 | P640 | M641 | A642 |
| P643 | N688 | E689 | P690 | T692 | Q731 | D733 | V752 | D756 |
| S807 | Y809 | I812 | D815 | L828 | V830 | Q831 | E832 | M833 |
| T834 | I836 | V845 | S872 | Y915 | S916 | A922 | P923 | F925 |

Compounds and/or Compositions

In one aspect, the invention provides compositions that are useful in treating endocrine FGF-related diseases or disorders. In certain embodiments, the compositions of the invention prevent or minimize the binding of FGF23 to α-Klotho on the surface of a mammal's cell.

(a) The Invention Provides a Construct that Prevents or Minimizes Binding of α-Klotho to FGF23 and/or a FGFR on the Surface of a Mammal's Cell.

In one aspect, the invention provides a construct (such as, but not limited to, an antibody and/or recombinant peptide) that prevents or minimizes the binding of FGF23 to α-Klotho on the surface of a mammal's cell.

In certain embodiments, the construct recognizes at least one amino acid residue of α-Klotho that binds to FGF23, thus preventing α-Klotho binding to FGF23. In other embodiments, the construct recognizes and/or binds to one or more amino acids within the amino acid residues 34-981 in α-Klotho (SEQ ID NO:1).

In certain embodiments, the construct recognizes and/or binds to one or more amino acids selected from the group consisting of amino acids F377, Q378, E390, S391, P392, W417, F418, V419, S420, K429, Y432, Y433, K436, N530, Q639, P640, M641, A642, P643, N688, E689, P690, T692, Q731, D733, V752, D756, S807, Y809, I812, D815, L828, V830, Q831, E832, M833, T834, I836, V845, S872, Y915, S916, A922, P923, and F925 of SEQ ID NO:1 (see Table 1 and FIG. 4A).

In certain embodiments, the construct recognizes and/or binds to at least amino acid F377 of SEQ ID NO:1. In certain embodiments, the construct recognizes and/or binds to at least amino acid Q378 of SEQ ID NO:1. In certain embodiments, the construct recognizes and/or binds to at least amino acid E390 of SEQ ID NO:1. In certain embodiments, the construct recognizes and/or binds to at least amino acid S391 of SEQ ID NO:1. In certain embodiments, the construct recognizes and/or binds to at least amino acid P392 of SEQ ID NO:1. In certain embodiments, the construct recognizes and/or binds to at least amino acid W417 of SEQ ID NO:1. In certain embodiments, the construct recognizes and/or binds to at least amino acid F418 of SEQ ID NO:1. In certain embodiments, the construct recognizes and/or binds to at least amino acid V419 of SEQ ID NO:1. In certain embodiments, the construct recognizes and/or binds to at least amino acid S420 of SEQ ID NO:1. In certain embodiments, the construct recognizes and/or binds to at least amino acid K429 of SEQ ID NO:1. In certain embodiments, the construct recognizes and/or binds to at least amino acid Y432 of SEQ ID NO:1. In certain embodiments, the construct recognizes and/or binds to at least amino acid Y433 of SEQ ID NO:1. In certain embodiments, the construct recognizes and/or binds to at least amino acid K436 of SEQ ID NO:1. In certain embodiments, the construct recognizes and/or binds to at least amino acid N530 of SEQ ID NO:1. In certain embodiments, the construct recognizes and/or binds to at least amino acid Q639 of SEQ ID NO:1. In certain embodiments, the construct recognizes and/or binds to at least amino acid P640 of SEQ ID NO:1. In certain embodiments, the construct recognizes and/or binds to at least amino acid M641 of SEQ ID NO:1. In certain embodiments, the construct recognizes and/or binds to at least amino acid A642 of SEQ ID NO:1. In certain embodiments, the construct recognizes and/or binds to at least amino acid P643 of SEQ ID NO:1. In certain embodiments, the construct recognizes and/or binds to at least amino acid N688 of SEQ ID NO:1. In certain embodiments, the construct recognizes and/or binds to at least amino acid E689 of SEQ ID NO:1. In certain embodiments, the construct recognizes and/or binds to at least amino acid P690 of SEQ ID NO:1. In certain embodiments, the construct recognizes and/or binds to at least amino acid T692 of SEQ ID NO:1. In certain embodiments, the construct recognizes and/or binds to at least amino acid Q731 of SEQ ID NO:1. In certain embodiments, the construct recognizes and/or binds to at least amino acid D733 of SEQ ID NO:1. In certain embodiments, the construct recognizes and/or binds to at least amino acid V752 of SEQ ID NO:1. In certain embodiments, the construct recognizes and/or binds to at least amino acid D756 of SEQ ID NO:1. In certain embodiments, the construct recognizes and/or binds to at least amino acid S807 of SEQ ID NO:1. In certain embodiments, the construct recognizes and/or binds to at least amino acid Y809 of SEQ ID NO:1. In certain embodiments, the construct recognizes and/or binds to at least amino acid I812 of SEQ ID NO:1. In certain embodiments, the construct recognizes and/or binds to at least amino acid D815 of SEQ ID NO:1. In certain embodiments, the construct recognizes and/or binds to at least amino acid L828 of SEQ ID NO:1. In certain embodiments, the construct recognizes and/or binds to at least amino acid V830 of SEQ ID NO:1. In certain embodiments, the construct recognizes and/or binds to at least amino acid Q831 of SEQ ID NO:1. In certain embodiments, the construct recognizes and/or binds to at least amino acid E832 of SEQ ID NO:1. In certain embodiments, the construct recognizes and/or binds to at least amino acid M833 of SEQ ID NO:1. In certain embodiments, the construct recognizes and/or binds to at least amino acid T834 of SEQ ID NO:1. In certain embodiments, the construct recognizes and/or binds to at least amino acid I836 of SEQ ID NO:1. In certain embodiments, the construct recognizes and/or binds to at least amino acid V845 of SEQ ID NO:1. In certain embodiments, the construct recognizes and/or binds to at least amino acid S872 of SEQ ID NO:1. In certain embodiments, the construct recognizes and/or binds to at least amino acid Y915 of SEQ ID NO:1. In certain embodiments, the construct recognizes and/or binds to at least amino acid S916 of SEQ ID NO:1. In certain embodiments, the construct recognizes and/or binds to at least amino acid A922 of SEQ ID NO:1. In certain embodiments, the construct recognizes and/or binds to at least amino acid P923 of SEQ ID NO:1. In certain embodiments, the construct recognizes and/or binds to at least amino acid F925 of SEQ ID NO:1.

In another aspect, the invention provides a construct that prevents or minimizes the binding of α-Klotho to a FGFR on the surface of a mammal's cell.

In certain embodiments, the construct recognizes at least one amino acid residue of α-Klotho that binds to a FGFR, thus preventing α-Klotho binding to the FGFR. In other embodiments, the construct recognizes and/or binds to one or more amino acids within the extracellular region of human α-Klotho (amino acid residues 34-981 of SEQ ID NO:1), or a fragment thereof. In yet other embodiments, the construct recognizes and/or binds to one or more amino acids within the fragment of the extracellular region of human α-Klotho comprising the amino acid residues 534-571 of SEQ ID NO:1.

As will be understood by one skilled in the art, any antibody that may recognize and specifically bind to FGF23/FGFR/α-Klotho is useful in the present invention. The invention should not be construed to be limited to any one type of antibody, either known or heretofore unknown, provided that the antibody can specifically bind to FGF23/FGFR/α-Klotho, and prevent or minimize binding of α-Klotho to FGF23 and/or FGFR. Methods of making and using such antibodies are well known in the art. For example, the generation of polyclonal antibodies may be accomplished by inoculating the desired animal with the antigen and isolating antibodies which specifically bind the antigen therefrom. Monoclonal antibodies directed against full length or peptide fragments of a protein or peptide may be prepared using any well-known monoclonal antibody preparation procedures, such as those described, for example, in Harlow et al. (1989, Antibodies, A Laboratory Manual, Cold Spring Harbor, N.Y.) and in Tuszynski et al. (1988, Blood 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein. However, the invention should not be construed as being limited solely to methods and compositions including these antibodies, but should be construed to include other antibodies, as that term is defined elsewhere herein.

In some instances, it is desirable to prepare monoclonal antibodies from various mammalian hosts, such as rodents (e.g., mice), primates (e.g., humans), etc. Descriptions of techniques for preparing such monoclonal antibodies are well known and are described, for example, in Harlow et al., ANTIBODIES: A LABORATORY MANUAL, COLD SPRING HARBOR LABORATORY, Cold Spring Harbor, N.Y. (1988); Harlow et al., USING ANTIBODIES: A LABORATORY MANUAL, (Cold Spring Harbor Press, New York, 1998); Breitling et al., RECOMBINANT ANTIBODIES (Wiley-Spektrum, 1999); and Kohler et al., 1997 Nature 256: 495-497; U.S. Pat. Nos. 5,693,762; 5,693,761; 5,585,089; and 6,180,370.

Nucleic acid encoding an antibody obtained using the procedures described herein may be cloned and sequenced using technology that is available in the art, and is described, for example, in Wright et al. (Critical Rev. Immunol. 1992, 12:125-168) and the references cited therein. Further, the antibody of the invention may be “humanized” using the technology described in Wright et al. (supra) and in the references cited therein, and in Gu et al. (Thrombosis and Hematocyst 1997, 77:755-759).

Alternatively, antibodies may be generated using phage display technology. To generate a phage antibody library, a cDNA library is first obtained from mRNA that is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, New York).

Bacteriophage that encode the desired antibody may be engineered such that the protein is displayed on the surface thereof in such a manner that it is available for binding to its corresponding binding protein, e.g., the antigen against which the antibody is directed. Thus, when bacteriophage that express a specific antibody are incubated in the presence of a cell that expresses the corresponding antigen, the bacteriophage will bind to the cell. Bacteriophage that do not express the antibody will not bind to the cell. Such panning techniques are well known in the art and are described for example, in Wright et al. (Critical Rev. Immunol. 1992, 12:125-168).

Processes such as those described herein have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage that display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage that encode the Fab portion of an antibody molecule. However, the invention should not be construed to be limited solely to the generation of phage encoding Fab antibodies. Rather, phage that encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J Mol Biol 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al., 1995, J Mol Biol 248:97-105).

The invention encompasses polyclonal, monoclonal, synthetic antibodies, and the like. One skilled in the art would understand, based upon the disclosure provided herein, that the crucial feature of the antibody of the invention is that the antibody specifically binds with FGF23 and/or α-Klotho.

In yet another aspect, the invention provides a soluble construct that is capable of sequestering α-Klotho and/or FGF23 on the surface of a mammal's cell.

In certain embodiments, the invention provides a soluble construct comprising a FGF23 polypeptide that is capable of binding to and sequestering α-Klotho on the surface of a mammal's cell. In certain embodiments, the FGF23 polypeptide comprises the amino acid residues 180-251 of SEQ ID NO:5 (FGF23_{CT}). FGF23_{CT} can be fused with another polypeptide, such as but not limited to a stability enhancing domain, such as but not limited to albumin, thioredoxin, glutathione S-transferase (GST), or a Fc region of an antibody. In certain embodiments, FGF23_{CT} and the stability enhancing domain are linked through a polypeptide comprising about 1-18 amino acids, 1-17 amino acids, 1-16 amino acids, 1-15 amino acids, 1-14 amino acids, 1-13 amino acids, 1-12 amino acids, 1-11 amino acids, 1-10 amino acids, 1-9 amino acids, 1-8 amino acids, 1-7 amino acids, 1-6 amino acids, 1-5 amino acids, 1-4 amino acids, 1-3 amino acids, 1-2 amino acids, or a single amino acid.

In yet another aspect, the invention provides a soluble construct comprising a α-Klotho polypeptide that is capable of binding to and sequestering FGF23. In certain embodiments, the α-Klotho polypeptide comprises the extracellular region of human α-Klotho (amino acid residues 34-981 of SEQ ID NO:1), or a fragment thereof. In other embodiments, the α-Klotho polypeptide comprises amino acid residues 377-925 of SEQ ID NO:1, or a fragment thereof. The α-Klotho polypeptide can be fused with another polypeptide, such as but not limited to a stability enhancing domain, such as but not limited to albumin, thioredoxin, glutathione S-transferase (GST), or a Fc region of an antibody. In certain embodiments, the α-Klotho polypeptide and the stability enhancing domain are linked through a polypeptide comprising 1-18 amino acids, 1-17 amino acids, 1-16 amino acids, 1-15 amino acids, 1-14 amino acids, 1-13 amino acids, 1-12 amino acids, 1-11 amino acids, 1-10 amino acids, 1-9 amino acids, 1-8 amino acids, 1-7 amino acids, 1-6 amino acids, 1-5 amino acids, 1-4 amino acids, 1-3 amino acids, 1-2 amino acids, or a single amino acid.

In yet other embodiments, the invention provides a soluble construct comprising a α-Klotho polypeptide that is capable of binding to FGFRs. In certain embodiments, the α-Klotho polypeptide comprises the extracellular region of human α-Klotho (the amino acid residues 34-981 of SEQ ID NO:1), or a fragment thereof. In other embodiments, the fragment of the extracellular region of human β-Klotho comprises the amino acid residues 534-571 of SEQ ID NO:1. The α-Klotho polypeptide can be fused with another polypeptide, such as but not limited to a stability enhancing domain, such as but not limited to albumin, thioredoxin, glutathione S-transferase (GST), or a Fc region of an antibody. In certain embodiments, the α-Klotho polypeptide and the stability enhancing domain are linked through a polypeptide comprising 1-18 amino acids, 1-16 amino acids, 1-14 amino acids, 1-12 amino acids, 1-10 amino acids, 1-8 amino acids, 1-6 amino acids, 1-5 amino acids, 1-4 amino acids, 1-3 amino acids, 1-2 amino acids or a single amino acid.

(b) The Invention Provides a Composition that Binds to α-Klotho and Induces Dimerization/Sequestration of α-Klotho.

In one aspect, the invention provides a soluble construct comprising a FGF23 C-terminus polypeptide that binds to α-Klotho and induces α-Klotho's dimerization. In certain embodiments, the FGF23 C-terminus polypeptide has the same sequence as the corresponding wild-type FGF23 fragment. In other embodiments, the FGF23 C-terminus polypeptide has at least one site-directed mutation away from the corresponding wild-type FGF23 fragment. In certain embodiments, the FGF23 C-terminus polypeptide comprises FGF23_{CT} (corresponding to the amino acid residues 180-251 of SEQ ID NO:5) or any site-directed mutant thereof. The FGF23 polypeptide can be fused with another polypeptide, such as but not limited to a stability enhancing domain, such as but not limited to albumin, thioredoxin, glutathione S-transferase, or a Fc region of an antibody. In certain embodiments, the FGF23 polypeptide and the stability enhancing domain are linked through a polypeptide comprising 1-18 amino acids, 1-16 amino acids, 1-14 amino acids, 1-12 amino acids, 1-10 amino acids, 1-8 amino acids, 1-6 amino acids, 1-5 amino acids, 1-4 amino acids, 1-3 amino acids, 1-2 amino acids or a single amino acid.

The compounds included in the compositions of the invention may form salts with acids, and such salts are included in the present invention. In certain embodiments, the salts are pharmaceutically acceptable salts. The term “salts” embraces addition salts of free acids that are useful within the methods of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, O-hydroxybutyric, salicylic, galactaric and galacturonic acid.

### Methods

In one aspect, the invention includes a method of treating or preventing a disease or disorder in a subject in need thereof.

In certain embodiments, the method comprises administering to the subject a therapeutically effective amount of a construct that prevents or minimizes binding of FGF23 and/or FGFR to α-Klotho on the surface of a mammal's cell. Non-limiting examples of diseases or disorders treated or prevented by the method includes various types of hypophosphatemia, such as, but not limited to, X-linked hypophosphatemia (XLH), autosomal recessive hypophosphatemic rickets 1 (ARHR1), hypophosphatemic rickets 2 (ARHR2), and autosomal dominant hypophatemic rickets (ADHR). Further non-limiting examples of diseases or disorders treated or prevented by the method includes tumor-induced osteomalacia (TIO). As the level of FGF23 is highly increased in patients suffering from Chronic Kidney Disease (CKD), inhibitors of α-Klotho or FGF23 can also be used for treatment of CKD patients.

In certain embodiments, the construct comprises a recombinant peptide and/or an antibody, and combinations thereof. In other embodiments, the antibody comprises at least one antibody selected from the group consisting of a polyclonal antibody, monoclonal antibody, humanized antibody, synthetic antibody, heavy chain antibody, human antibody, biologically active fragment of an antibody, and combinations thereof. In yet other embodiments, the subject is a mammal. In yet other embodiments, the mammal is human. In yet other embodiments, the construct is administered by an administration route selected from the group consisting of inhalational, oral, rectal, vaginal, parenteral, intracranial, topical, transdermal, pulmonary, intranasal, buccal, ophthalmic, intrathecal, and intravenous.

In certain embodiments, the subject is further administered at least one additional drug that treats the disease and/or disorder. In other embodiments, the construct and the at least one additional drug are co-administered. In yet other embodiments, the construct and the at least one additional drug are co-formulated.

### Combination Therapies

The compounds and compositions identified using the methods described here are useful in the methods of the invention in combination with one or more additional compounds useful for treating the diseases or disorders contemplated herein. These additional compounds may comprise compounds identified herein or compounds, e.g., commercially available compounds, known to treat, prevent, or reduce the symptoms of the diseases or disorders contemplated herein.

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Pharmaceutical Compositions and Formulations

The invention also encompasses the use of pharmaceutical compositions of the invention to practice the methods of the invention.

Such pharmaceutical compositions may be provided in a form suitable for administration to a subject, and may comprise one or more pharmaceutically acceptable carriers, one or more additional ingredients, or some combination of these. The compositions of the invention may comprise a physiologically acceptable salt, such as a compound contemplated within the invention in combination with a physiologically acceptable cation or anion, as is well known in the art.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for inhalational, oral, rectal, vaginal, parenteral, topical, intracranial, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

### Administration/Dosing

The regimen of administration may affect what constitutes an effective amount. The therapeutic formulations may be administered to the patient either prior to or after the manifestation of symptoms associated with the disease or condition. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, such as a human, may be carried out using known procedures, at dosages and for periods of time effective to treat a disease or condition in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 50 mg/kg of body weight/per day. One of ordinary skill in the art would be able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of cancer in a patient.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physical taking all other factors about the patient into account.

Compounds of the invention for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments therebetween.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder in a patient.

The term “container” includes any receptacle for holding the pharmaceutical composition. For example, in certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Routes of Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, intracranial, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or diglycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

Controlled- or sustained-release formulations of a pharmaceutical composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In certain embodiments of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction and assaying conditions with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials & Methods:

Unless otherwise noted, all starting materials were obtained from commercial suppliers and used without purification.

Certain modeling experiments performed with β-Klotho are recited in U.S. Provisional Application No. 62/529,215, which is incorporated herein in its entirety by reference.

Homology models of the extracellular region of α-Klotho (sKLA) in the apo state and the FGF19- and FGF23-bound states were generated with the program MODELLER v9.15 (Sali and Blundell, 1993, J Mol. Biol. 234:779-815), using the crystal structure of FGF21_{CT}-bound β-Klotho (sKLB) as a modeling template.

The sequence of sKLA (residues 34-955) was aligned to the corresponding residues in sKLB (residues 53-969 in the FGF21-bound sKLB crystal structure) using Clustal Omega (Sievers et al., 2011, Mol. Syst. Biol. 7:539) with the default settings. The sequences of FGF19_{CT} (residues 192-216) and FGF23_{CT} (residues 182-205) were aligned to the template FGF21_{CT} structure using PROMALS3D (Pei & Grishin, 2014, Methods Mol. Biol. 1079:263-271).

For the apo sKLA model and each of the sKLA-FGF_{CT} complex models, 20 initial models were first generated, and subsequently refined with two cycles of optimization, including 300 iterations of conjugated gradients using a variable-target function and molecular dynamics with simulated annealing. The models were inspected visually using the open-source molecular visualization program PyMOL.

To visualize the electrostatic potential of the protein surfaces om the sKLA models, the protein residues were first parameterized using the PDB2PQR (Dolinsky et al., 2004, Nucleic Acids Res. 32:W665-W667), and the electrostatic potentials were then calculated using the APBS (Baker et al., 2001, Proc. Natl. Acad. Sci. USA 98:10037-10041) through the PyMOL plugin Apbsplugin (pymolwiki dot org/index.php/Apbsplugin, retrieved on Oct. 12, 2017), using the default settings.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A non-natural soluble construct that prevents or minimizes the binding of a FGF receptor (FGFR) or FGF23 to α-Klotho, thus preventing FGFR activation.

2. The construct of claim 1, wherein the α-Klotho is on the surface of a mammal's cell.

3. The construct of claim 1, which is an antibody, nanobody, recombinant protein, or small molecule.

4. (canceled)

5. The construct of claim 3, wherein the antibody is selected from the group consisting of a polyclonal antibody, monoclonal antibody, humanized antibody, synthetic antibody, heavy chain antibody, human antibody, biologically active fragment of an antibody, and any combinations thereof.

6. The construct of claim 1, which recognizes and binds to at least one of the following:
(a) at least one amino acid residue of FGF23 that binds to α-Klotho, thus preventing FGF23 binding to α-Klotho;
(b) at least one amino acid residue of α-Klotho that binds to FGF23, thus preventing α-Klotho binding to FGF23;
(c) at least one amino acid residue of α-Klotho that binds to a FGFR, thus preventing α-Klotho binding to the FGFR.

7. (canceled)

8. The construct of claim 6, wherein the construct in (b) recognizes or binds to one or more amino acids within the amino acid residues 377-925 in α-Klotho (SEQ ID NO:1).

9. The construct of claim 8, wherein the construct in (b) recognizes or binds to one or more amino acids selected from the group consisting of F377, Q378, E390, S391, P392, W417, F418, V419, 5420, K429, Y432, Y433, K436, N530, Q639, P640, M641, A642, P643, N688, E689, P690, T692, Q731, D733, V752, D756, S807, Y809, I812, D815, L828, V830, Q831, E832, M833, T834, I836, V845, 5872, Y915, S916, A922, P923, and F925 of SEQ ID NO:1.

10. (canceled)

11. The construct of claim 16, wherein the construct in (c) recognizes or binds to one or more amino acids within the extracellular region of human α-Klotho (amino acid residues 34-981 of SEQ ID NO:1).

12. The construct of claim 11, wherein the construct in (c) recognizes or binds to one or more amino acids within the fragment of the extracellular region of human α-Klotho comprising amino acid residues 534-571 of SEQ ID NO:1.

13. The construct of claim 1, comprising at least one of the following:
(a) a FGF23 polypeptide that is capable of binding to and sequestering α-Klotho on the surface of a mammal's cell;
(b) a α-Klotho polypeptide that is capable of binding to and sequestering FGF23;
(c) a α-Klotho polypeptide that is capable of binding to a FGFR.

14. The construct of claim 13, which comprises amino acid residues 180-251 of SEQ ID NO:3 (FGF23_{CT}), or a fragment thereof.

15. (canceled)

16. The construct of claim 13 wherein in (b) the α-Klotho polypeptide comprises the extracellular region of human α-Klotho (amino acids 34-981 of SEQ ID NO:1), or a fragment thereof.

17. The construct of claim 16, wherein the α-Klotho polypeptide comprises amino acids 377-925 of SEQ ID NO:1, or a fragment thereof.

18. (canceled)

19. The construct of claim 13, wherein in (c) the construct comprises the extracellular region of human α-Klotho (amino acid residues 34-981 of SEQ ID NO:1), or a fragment thereof.

20. The construct of claim 19, which comprises amino acid residues 534-571 of SEQ ID NO:1, or a fragment thereof.

21. The construct of claim 1, which is fused to a stability enhancing domain.

22. The construct of claim 21, wherein the stability enhancing domain comprises albumin, thioredoxin, glutathione S-transferase, or a Fc region of an antibody.

23. The construct of claim 21, wherein the polypeptide and the stability enhancing domain are linked through a polypeptide comprising about 1-18 amino acids.

24. A soluble construct comprising a FGF23 polypeptide that binds to α-Klotho more tightly than wild-type FGF23 and elicits enhanced biological activity as compared to wild-type FGF23.

25. The construct of claim 24, wherein the FGF23 polypeptide has at least one mutation in its C-terminal domain.

26. The construct of claim 24, which is fused to a stability enhancing domain.

27. The construct of claim 26, wherein the stability enhancing domain comprises albumin, thioredoxin, glutathione S-transferase, or a Fc region of an antibody.

28. The construct of claim 26, wherein the polypeptide and the stability enhancing domain are linked through a polypeptide comprising about 1-18 amino acids.

29. A construct that simultaneously binds to an exposed epitope on FGF23_{CT} and an exposed epitope on α-Klotho in a FGF23_{CT}-α-Klotho complex, stabilizing the FGF23_{CT}-α-Klotho complex formation and eliciting enhanced biological activity as compared to wild-type FGF23.

30. The construct of claim 29, which is an antibody, nanobody, recombinant protein, or small molecule.

31. (canceled)

32. The construct of claim 29, wherein the antibody is selected from the group consisting of a polyclonal antibody, monoclonal antibody, humanized antibody, synthetic antibody, heavy chain antibody, human antibody, biologically active fragment of an antibody, and any combinations thereof.

33. A construct comprising a FGF23 polypeptide fused to a α-Klotho binder, wherein the construct has FGF23 stimulatory activities.

34. A method of treating or ameliorating preventing endocrine FGF-related diseases or disorders in a mammal in need thereof, the method comprising administering to the mammal a therapeutically effective amount of a construct that modulates interaction of FGF23 with α-Klotho on the surface of a cell of the mammal.

35. The method of claim 34, wherein the construct prevents or minimizes binding of FGF23 to α-Klotho on the surface of the mammal's cell.

36. The method of claim 35, wherein the disease or disorder includes hypophosphatemia or tumor-induced osteomalacia.

37. The method of claim 34, wherein the construct binds more tightly than wild-type FGF23 to α-Klotho on the surface of the mammal's cell.

38. The method of claim 34, wherein the mammal is human.

39. The method of claim 34, wherein the construct is administered by an administration route selected from the group consisting of inhalational, oral, rectal, vaginal, parenteral, intracranial, topical, transdermal, pulmonary, intranasal, buccal, ophthalmic, intrathecal, and intravenous.

40. The method of claim 34, wherein the mammal is further administered at least one additional drug that treats or prevents the disease or disorder.

41. The method of claim 40, wherein the construct and the at least one additional drug are co-administered or co-formulated.

42. (canceled)


SEQUENCE LISTING (8 sequences)

SEQ ID NO: 1 - protein, 1012 aa, Homo sapiens
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFASIISLSLIFYYSKKGRRSYK

SEQ ID NO: 2 - protein, 1044 aa, Homo sapiens
MKPGCAAGSPGNEWIFFSTDEITTRYRNTMSNGGLQRSVILSALILLRAVTGFSGDGRAIWSKNPNFTPVNESQLFLYDTFPKNFFWGIGTGALQVEGSWKKDGKGPSIWDHFIHTHLKNVSSTNGSSDSYIFLEKDLSALDFIGVSFYQFSISWPRLFPDGIVTVANAKGLQYYSTLLDALVLRNIEPIVTLYHWDLPLALQEKYGGWKNDTIIDIFNDYATYCFQMFGDRVKYWITIHNPYLVAWHGYGTGMHAPGEKGNLAAVYTVGHNLIKAHSKVWHNYNTHFRPHQKGWLSITLGSHWIEPNRSENTMDIFKCQQSMVSVLGWFANPIHGDGDYPEGMRKKLFSVLPIFSEAEKHEMRGTADFFAFSFGPNNFKPLNTMAKMGQNVSLNLREALNWIKLEYNNPRILIAENGWFTDSRVKTEDTTAIYMMKNFLSQVLQAIRLDEIRVFGYTAWSLLDGFEWQDAYTIRRGLFYVDFNSKQKERKPKSSAHYYKQIIRENGFSLKESTPDVQGQFPCDFSWGVTESVLKPESVASSPQFSDPHLYVWNATGNRLLHRVEGVRLKTRPAQCTDFVNIKKQLEMLARMKVTHYRFALDWASVLPTGNLSAVNRQALRYYRCVVSEGLKLGISAMVTLYYPTHAHLGLPEPLLHADGWLNPSTAEAFQAYAGLCFQELGDLVKLWITINEPNRLSDIYNRSGNDTYGAAHNLLVAHALAWRLYDRQFRPSQRGAVSLSLHADWAEPANPYADSHWRAAERFLQFEIAWFAEPLFKTGDYPAAMREYIASKHRRGLSSSALPRLTEAERRLLKGTVDFCALNHFTTRFVMHEQLAGSRYDSDRDIQFLQDITRLSSPTRLAVIPWGVRKLLRWVRRNYGDMDIYITASGIDDQALEDDRLRKYYLGKYLQEVLKAYLIDKVRIKGYYAFKLAEEKSKPRFGFFTSDFKAKSSIQFYNKVISSRGFPFENSSSRCSQTQENTECTVCLFLVQKKPLIFLGCCFFSTLVLLLSIAIFQRQKRRKFWKAKNLQHIPLKKGKRVVS

SEQ ID NO: 3 - protein, 216 aa, Homo sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 4 - protein, 209 aa, Homo sapiens
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 5 - protein, 251 aa, Homo sapiens
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTRSAEDDSERDPLNVLKPRARMTPAPASCSQELPSAEDNSPMASDPLGVVRGGRVNTHAGGTGPEGCRPFAKFI

SEQ ID NO: 6 - protein, 27 aa, Homo sapiens
PQPPDVGSSDPLSMVGPSQGRSPSYAS

SEQ ID NO: 7 - protein, 28 aa, Homo sapiens
SSPLETDSMDPFGLVTGLEAVRSPSFEK

SEQ ID NO: 8 - protein, 26 aa, Homo sapiens
SAEDDSERDPLNVLKPRARMTPAPAS